(19) United States
(12) Patent Application Publication
(10) Pub. No.: US 2011/0313282 A1
(43) Pub. Date: Dec. 22, 2011

(54) Ultrasound for Surgical Cranial Applications

(75) Inventors: Bruce M. Frankel, Mount Pleasant, SC (US); Randall R. Baraso, Mount Pleasant, SC (US); Kazutoshi Tsuchida, Tokyo (JP); Hiroaki Wakabayashi, Tokyo (JP)

(73) Assignee: ALOKA COMPANY, LTD., Wallingford, CT (US)

(21) Appl. No.: 13/087,648

(22) Filed: Apr. 15, 2011

Related U.S. Application Data
(60) Provisional application No. 61/324,845, filed on Apr. 16, 2010.

(51) Int. Cl. A61B 8/00

(52) U.S. Cl. 600/424

(57) ABSTRACT

The present disclosure relates generally to equipment and procedures in the field of surgery and/or diagnostics and, more particularly, to instruments, systems, and methods for undertaking surgical and/or diagnostic procedures that involve and/or are in proximity to the brain, e.g., cranial procedures and/or surgery. The disclosed devices generally include a handle member and an elongated probe that includes an ultrasound transducer. The devices may be used in conjunction with K-wires/guidewires, tubular members, e.g., EVD catheters and/or ventricular drains, endoscopes/cameras, and accessory items such as curettes, probes, knives, suction devices, scissors, cautery units, forceps, grasping devices and the like.

### CROSS-REFERENCE TO RELATED APPLICATIONS

This application claims the benefit of U.S. Provisional App. Ser. No. 61/324,845 filed Apr. 16, 2010, the entire contents of which is herein incorporated by reference in its entirety.

### BACKGROUND

1. Technical Field

The present disclosure relates generally to equipment and procedures in the field of surgery and/or diagnostics and, more particularly, to instruments, systems, and methods for undertaking surgical and/or diagnostic procedures that involve and/or are in proximity to the brain, e.g., cranial applications.

2. Background Art

Surgical and diagnostic procedures that involve and/or are in proximity to the brain require significant care to minimize the risk of inadvertent damage/injury to surrounding anatomical structures. For example, in pituitary surgical procedures, it is important to minimize the risk of injury to surrounding anatomical structures, e.g., the cavernous sinus contents. Surgical experience is valuable in reducing the risk of inadvertent injury. In addition, visualization techniques that employ microscopic, endoscopic and/or neuro-navigational equipment have been used to reduce injury risk.

However, despite prior efforts to reduce injury risk in such surgical/diagnostic procedures, a need remains for improved instruments, systems, and methods that facilitate desired surgical and/or diagnostic objectives, while minimizing the risk of injury to surrounding structures. In addition, a need remains for instruments, systems, and methods that fulfill the noted objective(s) through designs and techniques that are easily understood and implemented by surgical personnel.

These and other needs are satisfied by the instruments, systems and methods disclosed herein, as will be apparent from the detailed description which follows, particularly when read in conjunction with the figures appended hereto.

### SUMMARY

According to the present disclosure, advantageous instruments, systems, and methods are provided for undertaking surgical and/or diagnostic procedures that involve and/or are in proximity to the brain, e.g., cranial procedures and/or applications. In a first exemplary embodiment, the disclosed instrument, system and method generally includes a handle member that defines a guide wire channel and an elongated probe that is adapted to mount with respect to (or otherwise cooperate with) the handle member. The elongated probe includes an ultrasound transducer positioned at or near a distal end thereof. In exemplary embodiments, the ultrasound transducer is directed in a perpendicular or substantially perpendicular orientation relative to the axis of the elongated probe, such that non-axial ultrasound imaging is facilitated. In alternative implementations, the ultrasound transducer is directed in an axial or substantially axial orientation relative to the axis of the elongated probe. The handle member of the disclosed instruments/systems generally cooperates with conventional cabling for communication to and with the elongated probe and, in particular, the distally-positioned ultrasound transducer.

In use, the handle member of the disclosed instrument/system is adapted to receive a K-wire (or other guidewire) through the guide channel defined therein. The K-wire/guidewire may take various forms and exhibit various characteristics. For example, the K-wire/guidewire may be substantially rigid or flexible and may include a sharp or blunt end. In addition, exemplary implementations of the present disclosure may include a K-wire/guidewire that is threaded, in whole or in part. The K-wire/guidewire generally extends axially alongside the elongated probe such that its distal end may be positioned in close proximity to the region under ultrasound imaging. Thus, in exemplary embodiments, the handle is configured and dimensioned such that a stepped geometry is defined. The guide channel is formed in the outwardly stepped region of the handle, such that a K-wire that passes through the guide channel can easily run alongside the elongated probe in a substantially linear fashion.

The elongated probe with associated K-wire/guidewire may be advantageously introduced to a desired anatomical region, e.g., into the cranium of a patient, with real-time ultrasound imaging of anatomical structures adjacent thereto. In this way, potential injuries associated with inadvertent contact of the K-wire/guidewire with adjacent anatomical structure(s)/feature(s) may be avoided. Once the distal end of the K-wire/guidewire is positioned in a desired location/region, the elongated probe may be withdrawn while leaving the K-wire/guidewire in place. Thereafter, additional instrumentation and/or assemblies may be introduced to the anatomical location/region using the K-wire/guidewire as a guide, e.g., an external ventricular drain (EVD) catheter or ventricular drain to relieve intracranial pressure and hydrocephalus.

In a second exemplary embodiment of the present disclosure, the disclosed instrument, system and method generally includes a handle member and an elongated probe that is adapted to mount with respect to (or otherwise cooperate with) the handle member. The elongated probe includes an ultrasound transducer positioned at or near a distal end thereof. The ultrasound transducer is typically directed in either a perpendicular or substantially perpendicular orientation relative to the axis of the elongated probe, such that non-axial ultrasound imaging is facilitated, or in axial (or substantial axial) alignment with the elongated probe, such that axially-directed ultrasound imaging is facilitated. The handle member of the disclosed instruments/systems generally cooperates with conventional cabling for communication to and with the elongated probe and, in particular, the distally-positioned ultrasound transducer.

In use, the elongated probe of the disclosed instrument/system is adapted to receive a tubular member, e.g., an EVD catheter or a ventricular drain, therearound for delivery thereof to a desired anatomical region/location. The EVD catheter/ventricular drain extends axially alongside the elongated probe and is configured and dimensioned so as to permit unobstructed ultrasound imaging. Thus, in exemplary embodiments of the present disclosure, the EVD catheter/ventricular drain includes an opening, channel, window or other structural feature that permits unobstructed ultrasound imaging from the ultrasound transducer, whether such ultrasound imaging is directed axially, transversely or at some other orientation relative to the elongated member. In alternative implementations, the distal end of the EVD catheter/ventricular drain is positioned proximal of the ultrasound transducer, thereby permitting unobstructed ultrasound imaging from the elongated probe in a desired axial/angular direction. Thus, the EVD catheter/ventricular drain (or other tubular/catheter structure) may be introduced to a desired anatomical region/location while ultrasound imaging ensures that injury to adjacent anatomical structures/features is avoided.

Accordingly, the elongated probe with associated EVD catheter/ventricular drain (or other tubular/catheter structure) may be advantageously introduced to a desired anatomical region, e.g., into the cranium of a patient, with real-time ultrasound imaging of anatomical structures adjacent thereto. In this way, potential injuries associated with inadvertent contact of the EVD catheter/ventricular drain (or other tubular/catheter structure) with adjacent anatomical structure(s)/feature(s) may be avoided. Once the distal end of the EVD catheter/ventricular drain reaches a desired location/region, the elongated probe may be withdrawn while leaving the EVD catheter/ventricular drain (or other tubular/catheter structure) in place to relieve intracranial pressure and hydrocephalus.

In a further exemplary embodiment of the present disclosure, the disclosed instrument, system and method generally includes a handle member and an elongated probe that is adapted to mount with respect to (or otherwise cooperate with) the handle member. The elongated probe includes an ultrasound transducer positioned at or near a distal end thereof. The ultrasound transducer is typically directed in a perpendicular or substantially perpendicular orientation, or in an axial or substantially axial direction, relative to the axis of the elongated probe. The handle member of the disclosed instruments/systems generally cooperates with conventional cabling for communication to and with the elongated probe and, in particular, the distally-positioned ultrasound transducer. In use, the elongated probe of the disclosed instrument/system may be introduced to a desired anatomical region/location and the associated ultrasound imaging may be used to evaluate blood flow and/or flow velocities, e.g., during cranial aneurysm procedures/surgery, vascular procedures/surgery, intra-cranial procedures/surgery, extra-cranial procedures/surgery, bypass procedures/surgery, tumor-related procedures/surgery, and the like.

In a fourth exemplary embodiment of the present disclosure, the disclosed instrument, system and method generally includes a handle member and an elongated probe that is adapted to mount with respect to (or otherwise cooperate with) the handle member. The elongated probe includes an ultrasound transducer positioned at or near a distal end thereof. The ultrasound transducer is typically directed in a perpendicular or substantially perpendicular orientation, or in an axial or substantially axial orientation, relative to the axis of the elongated probe. The elongated probe further includes one or more integrated and/or modular accessory item(s) positioned at (or near) and extending from a distal end thereof. The integrated/modular item(s) that may be associated with the disclosed elongated probe include such items as a curette, a probe, a knife, a suction device, a scissor, a cautery unit, forceps, a grasping device and the like. Thus, for example, a curette may be provided that generally defines a tissue cutting element which can be used, for example, to resect tissue, e.g., a tumor. The operation and use of other integrated/modular item(s) are known and understood by persons skilled in the art and are not discussed/described further herein. The handle member of the disclosed instruments/systems generally cooperates with conventional cabling for communication to and with the elongated probe and, in particular, the distally-positioned ultrasound transducer.

In use, the elongated probe may be advantageously introduced to a desired anatomical region with real-time ultrasound imaging, e.g., to localize the pituitary gland and surround structures. The elongated probe may include one or more integrated/modular items for use in the diagnostic/surgical procedure. Thus, for example, a curette with tissue cutting element may be used to resect tissue, e.g., to remove pituitary tumors while observing the extent of resection through ultrasound imaging. The disclosed device may also be used to explore for residual tumor and visualize cavernous sinus contents, e.g., using color and power Doppler functionalities.

According to the present disclosure, it is further contemplated that the disclosed instruments/systems may be used in conjunction with an endoscope and/or endoscopic camera, thereby permitting simultaneous ultrasound imaging and conventional viewing. Thus, the elongated member may be adapted to cooperate with an endoscopic element that transmits images for viewing by medical personnel, thereby augmenting the ultrasound imaging delivered by the ultrasound transducer associated with the elongated element. In addition, the disclosed elongated member may be include one or more fiducials (e.g., flats or notches) or other antennae that may allow for the handle member and/or elongated member to be monitored/viewed by conventional neuro-navigation systems. In this way, the disclosed devices/systems may be advantageously integrated into intra-operative navigation systems, such as brain lab or stealth systems, so that the disclosed device may serve as a pointer for intra-operative navigation systems while also giving real-time feedback using ultrasound, which optionally may be merged with pre-operative MRI or CT scans.

Additional features, functions and benefits associated with the disclosed devices, systems and methods will be apparent from the detailed description which follows. For example, the disclosed devices, systems and methods may be used in conjunction with conventional technologies, e.g., microscopic and/or endoscopic visualization, to further enhance clinical efficacy.

### BRIEF DESCRIPTION OF THE DRAWINGS

So that those having ordinary skill in the art to which the subject disclosure appertains will more readily understand how to construct and employ the systems, apparatus and methods of the subject disclosure, reference may be had to the drawings wherein:

FIG. 1 is a side view of an exemplary device in cooperation with a K-wire/guidewire according to the present disclosure;

FIG. 2 is a schematic depiction of the exemplary device of FIG. 1 showing guidance to a desired anatomical location/region;

FIG. 3 is a further schematic depiction in which the exemplary device of the present disclosure has been withdrawn from the anatomical location/region, and the K-wire/guidewire is used to guide an EVD catheter/ventricular drain to such anatomical location/region;

FIG. 4 is a further schematic depiction in which, as compared to the schematic depiction of FIG. 3, the K-wire/guidewire has now been withdrawn leaving the EVD catheter/ventricular drain in position;

FIG. 5 is a side view of an alternative exemplary device in cooperation with an EVD catheter/ventricular drain (shown in phantom) according to the present disclosure;

FIG. 6 is a schematic depiction of the exemplary device of FIG. 5 showing the device with EVD catheter/ventricular drain (shown in phantom) prior to anatomical introduction;

FIG. 7 is a further schematic depiction in which the exemplary device of the present disclosure is introduced to a desired the anatomical location/region with the EVD catheter/ventricular drain (shown in phantom) guided to such anatomical location/region thereupon;

FIG. 8 is a further schematic depiction in which, as compared to the schematic depiction of FIG. 7, the elongated probe is being withdrawn leaving the EVD catheter/ventricular drain in position;

FIG. 9 is a further schematic depiction in which, as compared to the schematic depiction of FIG. 8, the elongated probe is fully withdrawn and the EVD catheter/ventricular drain remains in position;

FIG. 10 is a side view of a still further alternative exemplary device according to the present disclosure;

FIG. 11 is a schematic depiction of the exemplary device of FIG. 10 showing the device positioned at a desired anatomical region/location;

FIG. 12 is a side view of an additional alternative exemplary device according to the present disclosure;

FIG. 13 is a partial view of an exemplary curette that may be associated with the device of FIG. 12;

FIG. 14 is a schematic depiction of the exemplary device of FIG. 12 showing the device positioned at a desired anatomical region/location;

FIG. 15 is a side elevational view of an exemplary embodiment of a device similar to the instrument of FIG. 1 in accordance with the present disclosure for use in conjunction with a K-wire(s)/guidewire(s) (that may be introduced through alternative channels);

FIG. 16 is a side elevational view of another exemplary embodiment of a device in accordance with the present disclosure for use in conjunction with a K-wire/guidewire;

FIG. 17 is a side perspective view of another exemplary embodiment of a device in accordance with the present disclosure for use in conjunction with a K-wire/guidewire; and

FIGS. 17A and 17B are side elevational views of alternative elongated probes having at least one hollow receiver member mounted thereto for use with a device similar to the device of FIG. 17.

### DESCRIPTION OF EXEMPLARY EMBODIMENT(S)

In accordance with embodiments of the present disclosure, advantageous medical diagnostic and surgical instruments, systems, and methods are provided for use during a broad variety of applications and procedures within the cranium and/or in connection with and/or in proximity to the brain.

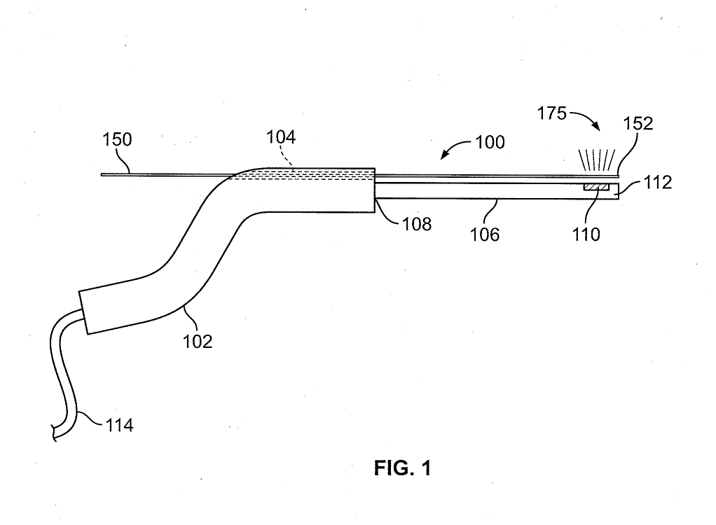

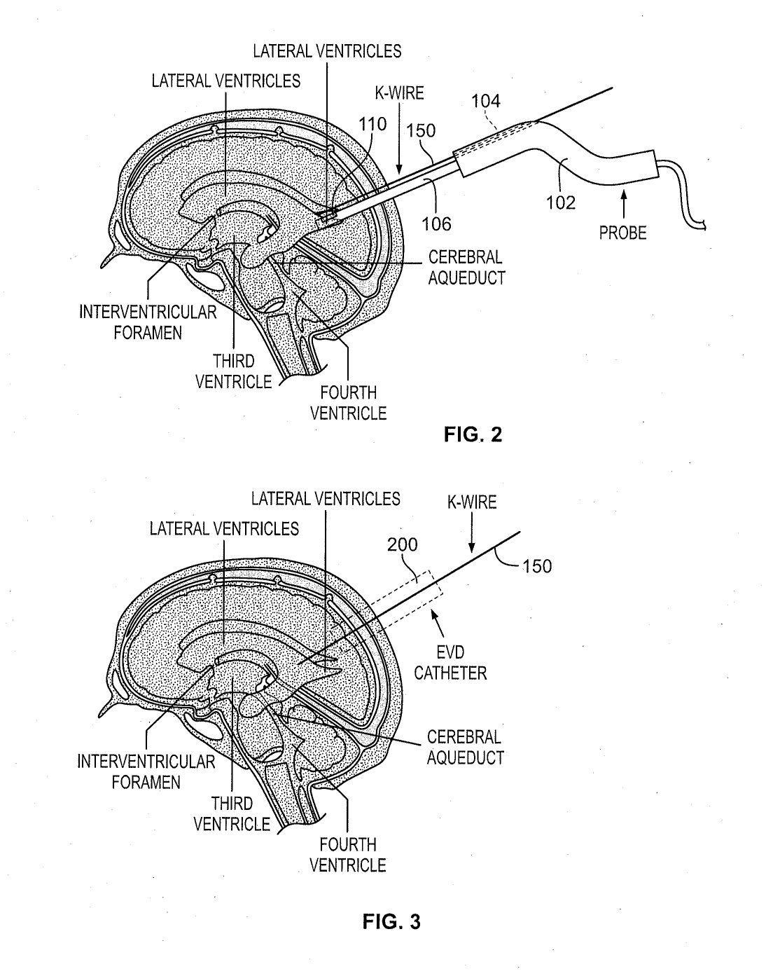

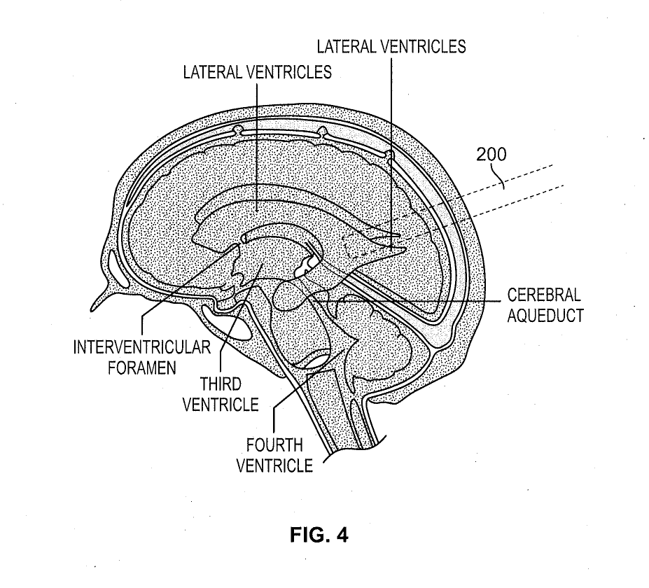

Referring now to FIGS. 1-4, a first exemplary device 100 generally includes a handle member 102 that defines guide wire channel 104 and an elongated probe 106 that is adapted to mount with respect to (or otherwise cooperate with) the handle member 102. In exemplary embodiments of the present disclosure, the elongated probe 106 may be detachably mounted with respect to handle member 102, e.g., by way of a bayonet locking mechanism, in junction region 108. Appropriate electrical connections are generally made in the junction region 108 to facilitate electronic communications between the handle member 102 (and accessory componentry/power source) and the elongated probe 106 (and associated operative functionalities).

The elongated probe 106 includes an ultrasound transducer 110 positioned at or near a distal end 112 thereof. In the embodiment depicted in FIGS. 1-4, the ultrasound transducer 110 is directed in a perpendicular or substantially perpendicular orientation relative to the axis of the elongated probe 106, such that non-axial ultrasound imaging is facilitated. However, the disclosed device may alternatively be provided with a ultrasound transducer that is positioned so as to be axially or substantially axially oriented with respect to the axis defined by elongated probe 106. The handle member 102 of the device 100 generally cooperates with conventional cabling 114 for communication to and with the elongated probe 106 and, in particular, the distally-positioned ultrasound transducer 110.

In use, the handle member 102 of device 100 is adapted to receive a K-wire/guidewire 150 (or other elongated structure) through the guide channel 104 defined therein. The K-wire/guidewire may take various forms and exhibit various properties, e.g., it may be sharp/blunt, rigid/flexible, threaded (in whole or in part), etc. The K-wire/guidewire 150 extends axially alongside the elongated probe 106 such that its distal end 152 may be positioned in close proximity to the region 175 under ultrasound imaging. Thus, in exemplary embodiments, the handle 102 is configured and dimensioned such that a stepped geometry is defined in the junction region 108. The guide channel 104 is formed in the outwardly stepped region of the handle 102, such that a K-wire/guidewire 150 that passes through the guide channel 104 can easily run alongside the elongated probe 106 in a substantially linear fashion.

The elongated probe 106 with associated K-wire/guidewire 150 may be advantageously introduced to a desired anatomical region, e.g., into the cranium of a patient, with real-time ultrasound imaging of anatomical structures adjacent thereto. In this way, potential injuries associated with inadvertent contact of the K-wire/guidewire 150 with adjacent anatomical structure(s)/feature(s) may be avoided. Once the distal end 152 of the K-wire/guidewire 150 is positioned in a desired location/region, the elongated probe 106 may be withdrawn while leaving the K-wire/guidewire 150 in place. Thereafter, additional instrumentation and/or assemblies may be introduced to the anatomical location/region using the K-wire/guidewire 150 as a guide, e.g., an external ventricular drain (EVD) catheter/ventricular drain 200 to relieve intracranial pressure and/or hydrocephalus.

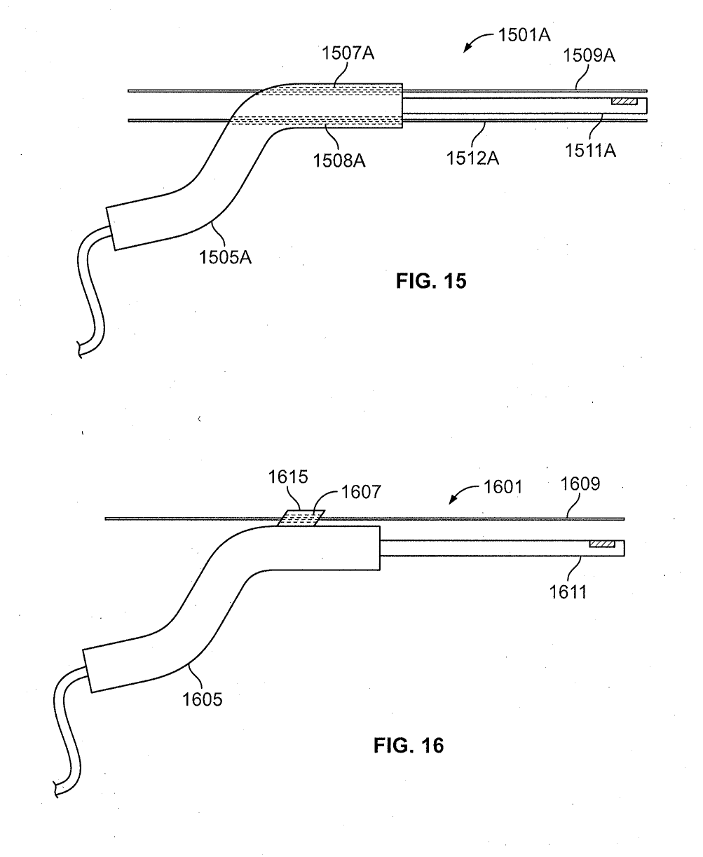

With reference to FIG. 15, an alternative device 1501A is shown. The device 1501A may be structurally and functionally similar to the device 100 discussed above with reference to FIG. 1, with certain additional features. In general, handle member 1505A of device 1501A defines first channel 1507A and second channel 1508A. In general, the channels 1507A and 1508A are formed in the handle member 1505A and extend therethrough. Both channels 1507A and 1508A are configured and dimensioned to receive a K-wire/guidewire, e.g., K-wire 1509A and/or K-wire 1512A, to permit the device 1501A to be slidably mounted thereto for purposes of guiding the device 1501 to a desired anatomical region, e.g., into the cranium of a patient, with real-time ultrasound imaging of anatomical structures adjacent thereto. Once the distal ends of the K-wires/guidewires 1509A and/or 1511A are positioned in a desired location/region, the elongated probe 1511A may be withdrawn while leaving K-wires/guidewires 1509A and/or 1511A in place, as described above. In one embodiment, the operator/surgeon would be free to select the channel 1509A and/or 1511A to be used for K-wire introduction.

Turning now to FIG. 16, a device 1601 in accordance with embodiments of the present disclosure is shown. The device 1601 may be structurally and functionally similar to the device 100 discussed above with reference to FIG. 1, with certain additional features. A channel 1607 is configured and dimensioned to receive a K-wire/guidewire 1609 to permit the device 1601 to be slidably mounted thereto for purposes of guiding the device 1601 to a desired anatomical region, e.g., into the cranium of a patient, the channel 1607 being formed in an extension 1615 of the handle member 1605 and extending past the handle member 1605.

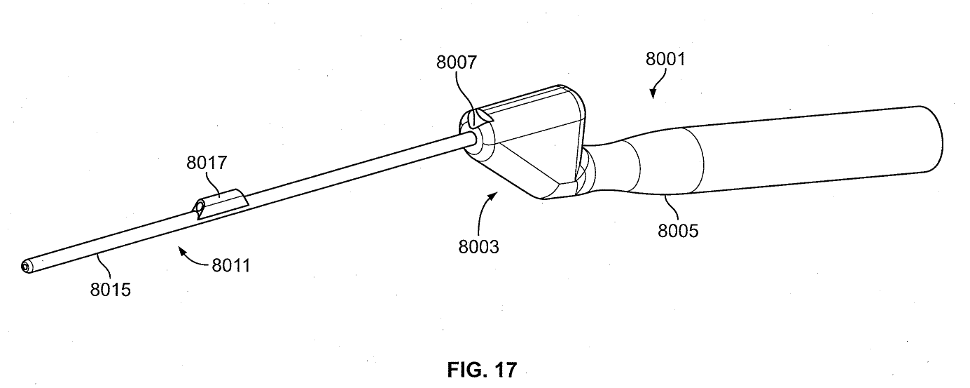

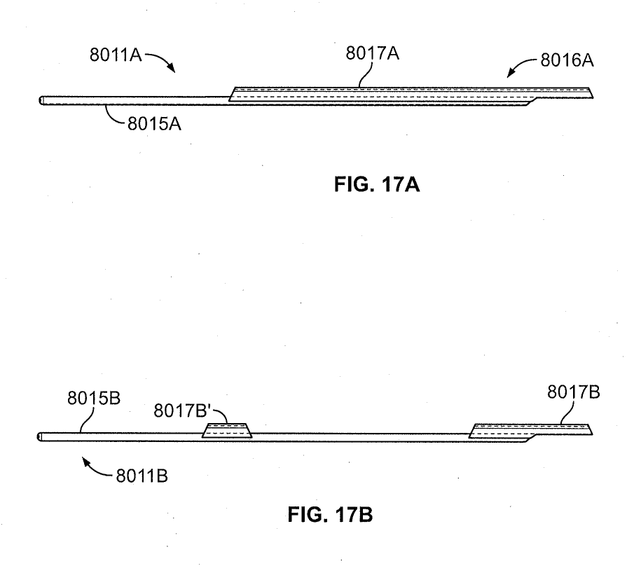

With reference to FIG. 17, a device 8001 in accordance with embodiments of the present disclosure is shown. Device 8001 may be structurally and functionally similar to the device 100 discussed above with reference to FIG. 1, with some differences. In general, device 8001 includes at least one hollow receiver member 8017 mounted with respect to longitudinal shaft 8015 of elongated probe 8011. In general, the at least one hollow receiver member 8017 is configured and dimensioned to receive a K-wire or guidewire or the like (e.g., a wire similar to K-wire 150 of FIG. 1) to permit the device 8001 to be slidably mounted thereto for purposes of guiding device 8001 to a desired anatomical region, e.g., into the cranium of a patient, with real-time ultrasound imaging of anatomical structures adjacent thereto. Once the distal end of the K-wire/guidewire (e.g., a wire similar to K-wire 150 of FIG. 1) is positioned in a desired location/region, the elongated probe 8011 may be withdrawn while leaving K-wire/guidewire in place, as described above.

The elongated probe 8011 typically includes an ultrasound transducer mounted to the longitudinal shaft 8015 proximate the distal end thereof, with the ultrasound transducer typically having an array of side-firing ultrasonic energy generation elements extending along the longitudinal shaft 8015 (similar to device 100 of FIG. 1 having ultrasound transducer 110). In one embodiment, the at least one hollow receiver member 8017 is positioned or mounted with respect to a distal portion of the longitudinal shaft 8015 of elongated probe 8011, although the present disclosure is not limited thereto. Rather, the at least one hollow receiver member 8017 may be positioned or mounted to any portion of the longitudinal shaft 8015 of elongated probe 8011.

In one embodiment and as shown in FIG. 17, handle 8003 of device includes a housing 8005, the housing 8005 including a channel 8007 formed therein, with the channel 8007 also configured and dimensioned to receive the K-wire or guidewire or the like that is received in hollow receiver member 8017 to permit the device 8001 to be slidably mounted thereto for purposes of guiding device 8001 to a desired anatomical region. In exemplary embodiments, channel 8007 extends through handle 8003.

Alternatively, channel 8007 may be formed in an extension of the housing 8005 of the handle 8003 (e.g., similar to extension 1615 of FIG. 16). In one embodiment, the longitudinal axis defined by channel 8007 is substantially the same as and/or is substantially aligned with the longitudinal axis defined by the at least one hollow receiver member 8017 (i.e., the same K-wire 150 would extend through channel 8007 and receiver member 8017). Alternatively, housing 8005 does not include channel 8007, and the K-wire or the like only travels through the at least one receiver member 8017 to permit the instrument 8001 to be slidably mounted thereto for guiding purposes.

In another embodiment and as depicted in FIG. 17A, an elongated probe 8011A for use with a device similar to instrument 8001 is depicted. At least one hollow receiver member 8017A is mounted with respect to longitudinal shaft 8015A of elongated probe 8011A and extends from a portion (e.g., a distal portion) of the shaft 8015A to a proximal end 8016A of shaft 8015A. In general, hollow receiver member 8017A is configured and dimensioned to receive a K-wire or guidewire or the like to permit the device (e.g., device 8001) to be slidably mounted thereto for guiding purposes.

In exemplary embodiments, the housing (e.g., housing similar to 8005) of the handle of the device for use with elongated probe 8011A may be configured and dimensioned to house and/or mount with respect to at least a portion of the proximal end 8016A of shaft 8015A. For example, at least a portion of the proximal end 8016A of shaft 8015A defines at least a portion of a channel through the housing of the handle of the device for use with probe 8011A. The housing of the handle of the device for use with elongated probe 8011A may or may not include a separate channel through the handle for use with the K-wire (e.g., separate from proximal end 8016A housed in the housing).

In another embodiment and as shown in FIG. 17B, an elongated probe 8011E for use with a device similar to instrument 8001 is depicted. Elongated probe 8011B includes a first hollow receiver member 8017B and a second hollow receiver member 8017B′, with the first and second hollow receiver members 8017B, 8017B′ being mounted with respect to longitudinal shaft 8015B of elongated probe 8011B. In general, first and second hollow receiver members 8017B, 8017B′ are configured and dimensioned to receive a K-wire or guidewire or the like (e.g., wire 150 of FIG. 1) to permit the device to be slidably mounted thereto for guiding purposes.

In one embodiment, first hollow receiver member 8017B is positioned or mounted with respect to a proximal end of probe 8011B, and second hollow receiver member 8017B′ is positioned or mounted with respect to a distal portion of probe 8011B, although the present disclosure is not limited thereto. The housing of the handle of the device for use with probe 8011B may be configured and dimensioned to house and/or mount with respect to at least a portion of first hollow receiver member 8017B. For example, at least a portion of first hollow receiver member 8017B defines at least a portion of a channel through the housing of the handle of the device for use with probe 8011B. The housing of the handle of the device for use with probe 8011B may or may not include a separate channel through the handle for use with the K-wire (i.e., separate from first hollow receiver member 8017B housed in the housing). In exemplary embodiments, the longitudinal axis defined by the first hollow receiver member 8017B is substantially the same as and/or is substantially aligned with the longitudinal axis defined by the second hollow receiver member 8017B′ (i.e., the same K-wire 150 would extend through first hollow receiver member 8017B and second hollow receiver member 8017B′).

Variations and modifications of the above-described devices are possible in accordance with embodiments of the present disclosure. In accordance with some such variations and modifications (not shown), the handle and the longitudinal shaft of the elongated probe (and/or the longitudinal shaft of the elongated probe and the hollow receiver members) are of unitary construction with respect to each other. Each of the above-described devices may be equipped with a cable assembly for carrying electrical signals to and from the ultrasound transducer in accordance with an ultrasonic imaging mode of use of the instrument, the cable assembly including a proximal end including an electrical connector for connecting the instrument to a corresponding ultrasound console and current carrying wires extending distally from the electrical connector to the ultrasound transducer at least partially via a corresponding interior conduit formed in and extending longitudinally along the longitudinal shaft of the ultrasound probe, as explained and described in U.S. Patent Publication No. 2011/0077525 and/or U.S. patent application Ser. No. 12/917,721, the entire contents of each being incorporated by reference herein. It is also noted that other variations and modifications are possible. Thus, the present disclosure provides, inter alia, advantageously integrated medical diagnostic instruments, systems incorporating such instruments, and methods of use of such instruments and systems for the benefit of such surgical practitioners and their patients.

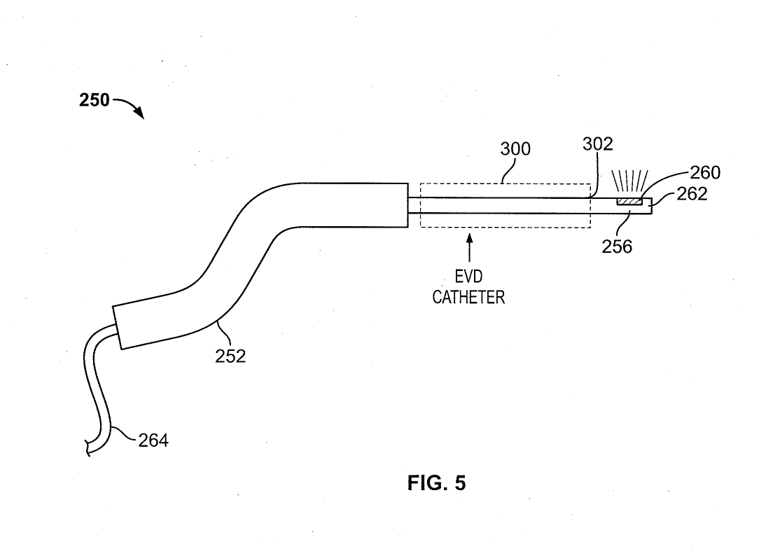

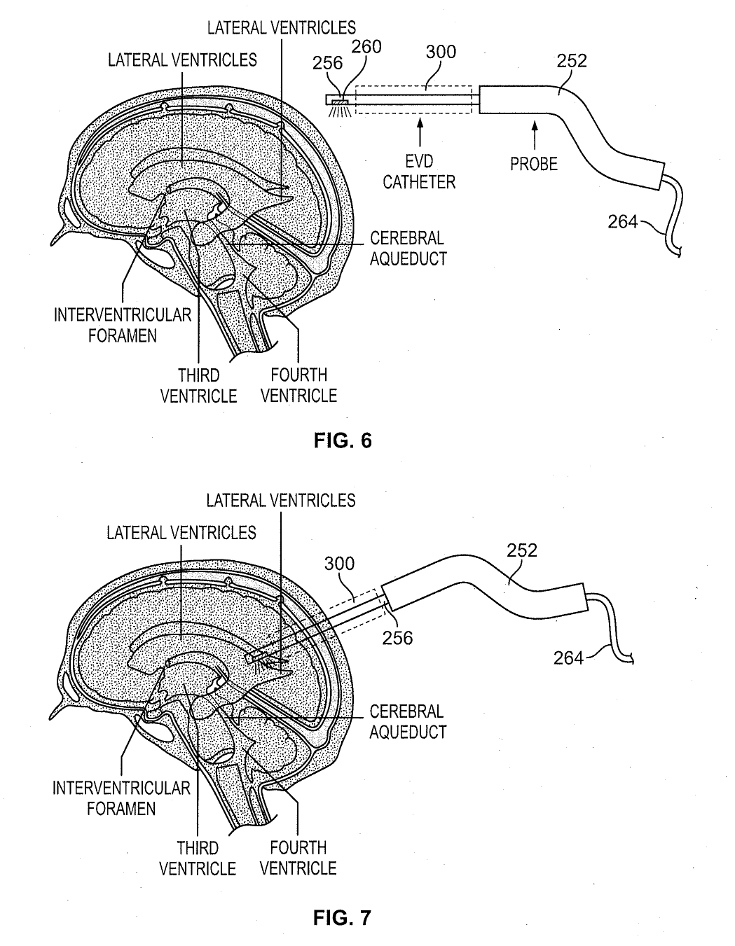

In another exemplary embodiment of the present disclosure, device 250 (FIG. 5) generally includes a handle member 252 and an elongated probe 256 that is adapted to mount with respect to (or otherwise cooperate with) the handle member 252, e.g., based on a bayonet locking mechanism. The elongated probe 256 includes an ultrasound transducer 260 positioned at or near a distal end 262 thereof. As described above, the ultrasound transducer 260 may be designed in a perpendicular/substantially perpendicular orientation relative to the axis of the elongated probe 256, such that non-axial ultrasound imaging is facilitated, or in an axial/substantially axial orientation relative to the axis of elongated probe 256. The handle member 252 of the device 250 generally cooperates with conventional cabling 264 for communication to and with the elongated probe 256 and, in particular, the distally-positioned ultrasound transducer 260.

In use, the elongated probe 256 of device 250 is adapted to receive a tubular member, e.g., an EVD catheter/ventricular drain 300, therearound for delivery thereof to a desired anatomical region/location. The EVD catheter/ventricular drain 300 extends axially alongside the elongated probe 256 such that its distal end 302 is positioned in close proximity to the ultrasound transducer. Thus, as depicted in the accompanying figures, the distal end 302 of the EVD catheter/ventricular drain 300 is positioned proximal of the ultrasound transducer 260, thereby permitting unobstructed ultrasound imaging from the elongated probe 256. In other exemplary embodiments of the present disclosure, however, the distal end 302 of the EVD catheter/ventricular drain 300 is provided with an opening, channel, window or other structural feature that permits unobstructed ultrasound imaging from the ultrasound transducer, whether such ultrasound imaging is directed axially, transversely or at some other orientation relative to the elongated member 256. Thus, the EVD catheter 300 (or other tubular/catheter structure) may be introduced to a desired anatomical region/location while ultrasound imaging ensures that injury to adjacent anatomical structures/features is avoided.

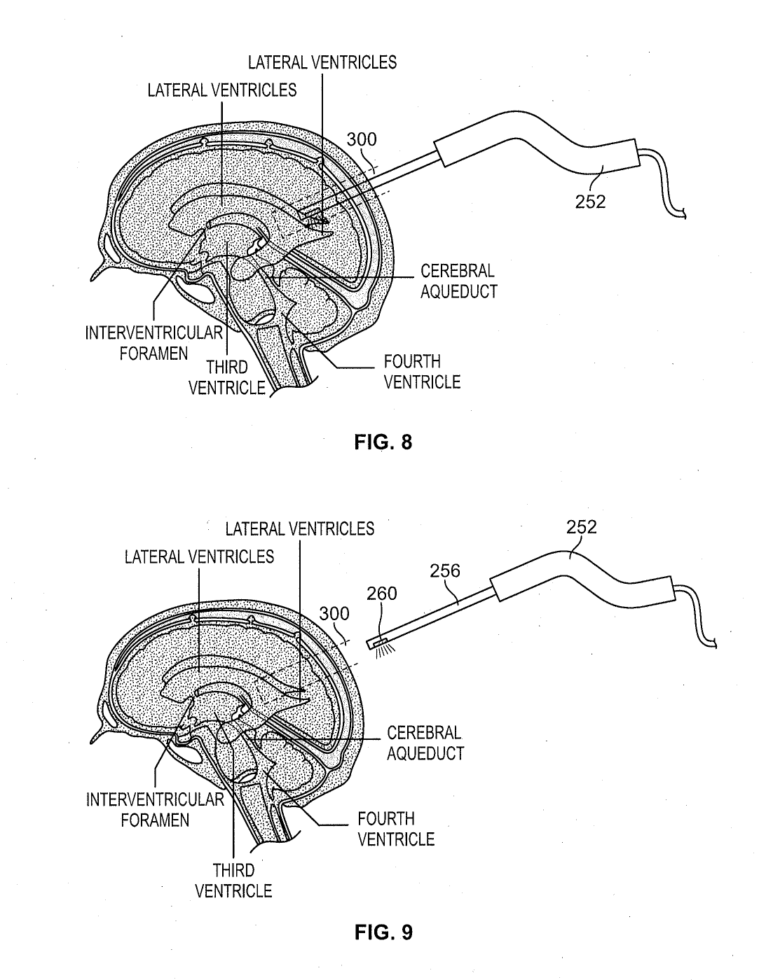

Accordingly, the elongated probe 256 with associated EVD catheter/ventricular drain 300 may be advantageously introduced to a desired anatomical region, e.g., into the cranium of a patient, with real-time ultrasound imaging of anatomical structures adjacent thereto. In this way, potential injuries associated with inadvertent contact of the EVD catheter 300 with adjacent anatomical structure(s)/feature(s) may be avoided. Once the distal end 302 of the EVD catheter/ventricular drain 300 reaches a desired location/region, the elongated probe 256 may be withdrawn while leaving the EVD catheter/ventricular drain 300 in place to relieve intracranial pressure and hydrocephalus (FIGS. 8-9).

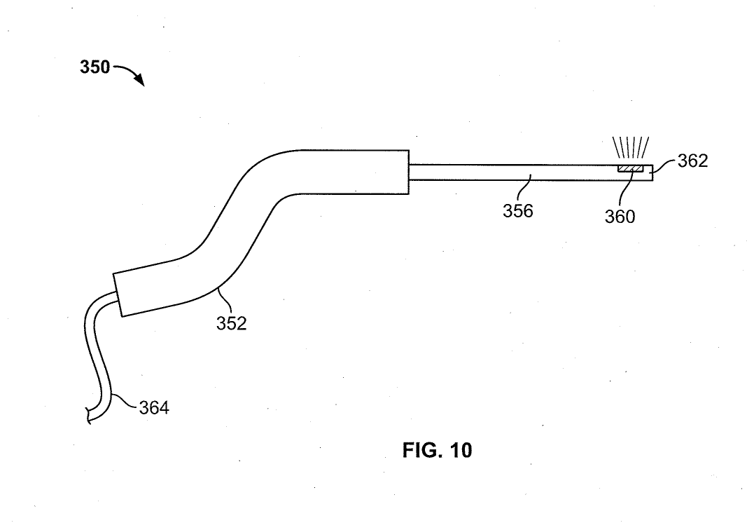

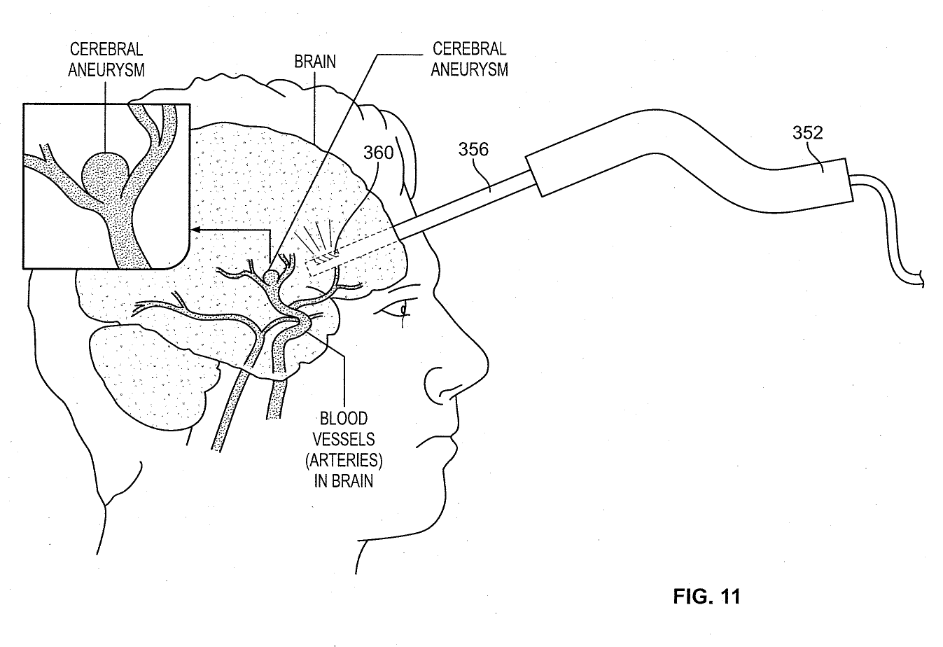

In a further exemplary embodiment of the present disclosure, device 350 (FIG. 10) generally includes a handle member 352 and an elongated probe 356 that is adapted to mount with respect to (or otherwise cooperate with) the handle member 352. The elongated probe 356 includes an ultrasound transducer 360 positioned at or near a distal end 362 thereof. As with previous embodiments disclosed herein, the ultrasound transducer 360 may be directed in a perpendicular or substantially perpendicular orientation relative to the axis of the elongated probe 356, such that non-axial ultrasound imaging is facilitated, or in an axial or substantially axially orientation relative to the axis of the elongated probe 356. The handle member 352 of device 350 generally cooperates with conventional cabling 364 for communication to and with the elongated probe 356 and, in particular, the distally-positioned ultrasound transducer 360. In use, the elongated probe 356 of device 350 may be introduced to a desired anatomical region/location and the associated ultrasound imaging may be used to evaluate blood flow and/or flow velocities, e.g., during cranial aneurysm procedures/surgery, vascular procedures/surgery, intra-cranial procedures/surgery, extra-cranial procedures/surgery, bypass procedures/surgery, tumor-related procedures/surgery, and the like.

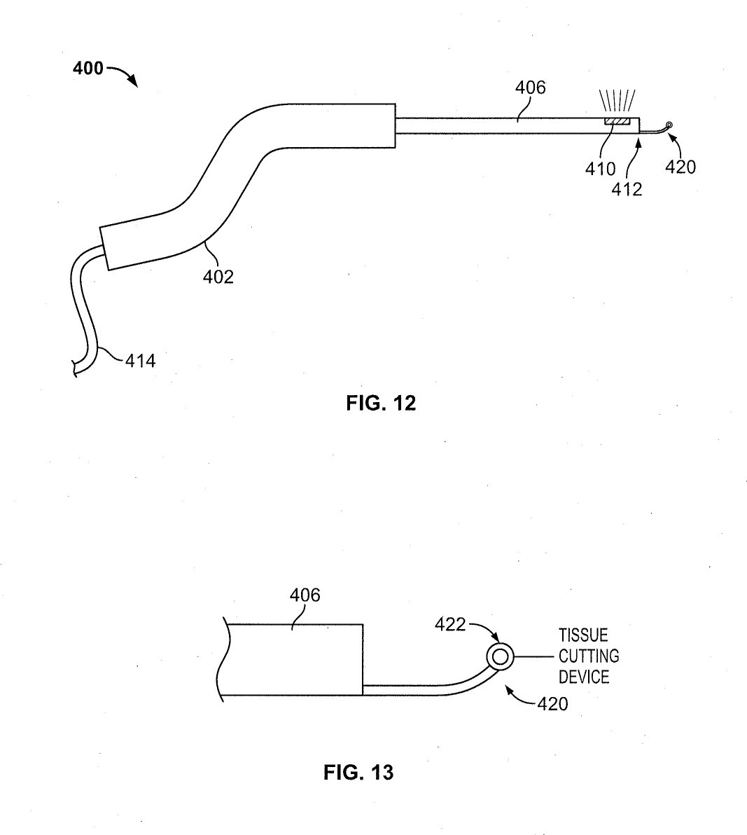

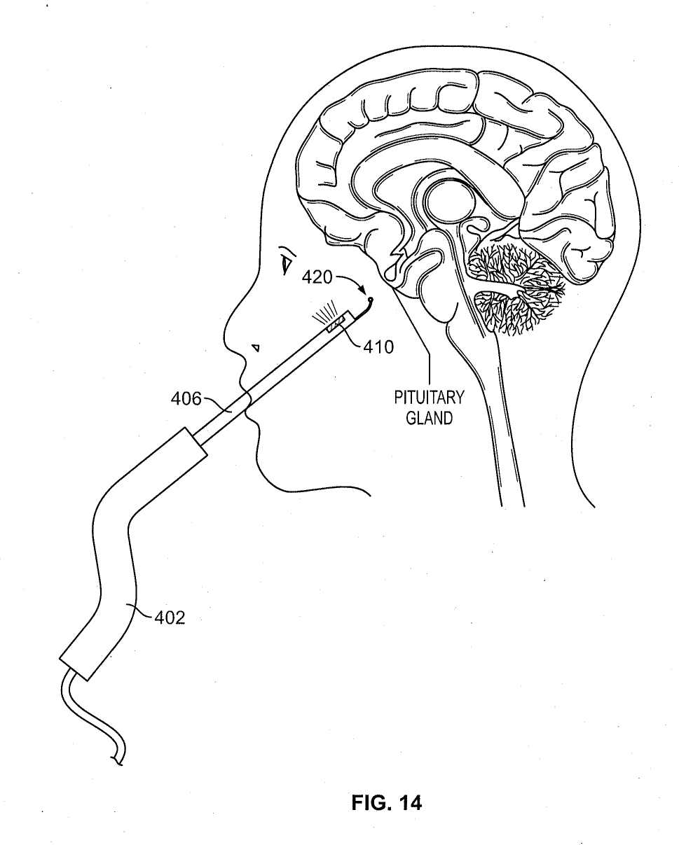

In another exemplary embodiment of the present disclosure, device 400 (FIG. 12) generally includes a handle member 402 and an elongated probe 406 that is adapted to mount with respect to (or otherwise cooperate with) the handle member 402, e.g., by way of a bayonet locking mechanism. The elongated probe 406 includes an ultrasound transducer 410 positioned at or near a distal end 412 thereof. The ultrasound transducer 410 may be directed in a perpendicular/substantially perpendicular orientation or an axial/substantially axial orientation relative to the axis of the elongated probe 406. The elongated probe 406 further includes an integrated or modular/interchangeable curette 420 positioned at (or near) and extending from a distal end 412 thereof. The curette 420 generally defines a tissue cutting element 422 which can be used, for example, to resect tissue, e.g., a tumor. The handle member 402 of device 400 generally cooperates with conventional cabling 414 for communication to and with the elongated probe 406 and, in particular, the distally-positioned ultrasound transducer 410.

Although the illustrated embodiment features a curette 420, the present disclosure is not limited thereby. For example, the elongated probe 406 may advantageously interact with one or more integrated and/or modular accessory item(s) positioned at (or near) and extending from a distal end 412 thereof. The integrated/modular item(s) that may be associated with the disclosed elongated probe include, for example, such items as a curette 420, a probe, a knife, a suction device, a scissor, a cautery unit, forceps, a grasping device and the like.

In use, the elongated probe 406 may be advantageously introduced to a desired anatomical region with real-time ultrasound imaging, e.g., to localize the pituitary gland 440 and surrounding structures. The curette 420 with tissue cutting element 422 may be used to resect tissue, e.g., to remove pituitary tumors while observing the extent of resection through ultrasound imaging. Alternatively, the elongated probe 406 may support alternative structures/elements, e.g., a probe, a knife, a suction device, a scissor, a cautery unit, forceps or a grasping device, that may be used to achieve desired clinical/diagnostic results. The disclosed device 400 may also be used to explore for residual tumor and visualize cavernous sinus contents, e.g., using color and power Doppler functionalities.

According to the present disclosure, the disclosed instruments/systems may be used in conjunction with an endoscope and/or endoscopic camera, thereby permitting simultaneous ultrasound imaging and conventional viewing. Thus, the elongated member may be adapted to cooperate with an endoscopic element that transmits images for viewing by medical personnel, thereby augmenting the ultrasound imaging delivered by the ultrasound transducer associated with the elongated element. In addition, the disclosed elongated member may include one or more fiducials (e.g., flats or notches) or other antennae that may allow for the handle member and/or elongated member to be monitored/viewed by conventional neuro-navigation systems. In this way, the disclosed devices/systems may be advantageously integrated into intra-operative navigation systems, such as brain lab or stealth systems, so that the disclosed device may serve as a pointer for intra-operative navigation systems while also giving real-time feedback using ultrasound, which optionally may be merged with pre-operative MRI or CT scans.

Although the systems, apparatus and methods have been described with respect to exemplary embodiments herein, it is apparent that modifications, variations, changes and/or enhancements may be made thereto without departing from the spirit or scope of the invention as defined by the appended claims. For example, as an alternative to the use of a side-firing ultrasound transducer as described hereinabove, and/or in addition thereto, one or more end-firing ultrasound transducers, and/or 360 degree ultrasound transducers may be employed, whether mounted with respect to the distal end of the longitudinal shaft of the associated ultrasound probe, adjacent thereto, or otherwise, for use as desired by the surgical practitioner. Accordingly, the present disclosure expressly encompasses all such modifications, variations, changes and/or enhancements.

Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A device for use in cranial procedures comprising:
a. a handle member;
b. an elongated probe extending from and mounted with respect to the handle member, the elongated probe supporting an ultrasound transducer for use in obtaining ultrasound images for use in determining the location of the elongated probe relative to surrounding anatomical structures or features.

2. A device according to claim 1, wherein the elongated probe is detachably connected relative to the handle member.

3. A device according to claim 1, wherein the handle member defines a guide channel that is sized and configured for receipt of a K-wire or guidewire.

4. A device according to claim 3, wherein the K-wire or guidewire is characterized by one or more of the following characteristics: flexibility, rigidity, sharpness, bluntness and threading.

5. A device according to claim 3, wherein the guide channel is sized and configured to permit the K-wire or guidewire to run alongside the elongated probe.

6. A device according to claim 1, wherein the ultrasound transducer is oriented in at least one of the following orientations: perpendicular or substantially perpendicular relative to the axis of the elongated probe, axial or substantially axial relative to the axis of the elongated probe, or angled relative to the axis of the elongated probe.

7. A device according to claim 1, wherein the elongated probe is sized and configured to releasably support a tubular member.

8. A device according to claim 7, wherein the tubular member is selected from the group consisting of an EVD catheter and a ventricular drain.

9. A device according to claim 8, wherein the EVD catheter or the ventricular drain defines an opening, channel, window or other structural feature that permits unobstructed ultrasound imaging from the ultrasound transducer.

10. A device according to claim 1, wherein the elongated probe is adapted to interact with member selected from the group consisting of a curette, a probe, a knife, a suction device, a scissor, a cautery unit, forceps and a grasping device.

11. A device according to claim 10, wherein at least one of the curette, probe, knife, suction device, scissor, cautery unit, forceps and grasping device is adapted to be detachably connected to the elongated probe.

12. A device according to claim 1 further comprising at least one hollow receiver member mounted with respect to the elongated probe;
wherein the at least one hollow receiver member is configured and dimensioned to receive a K-wire or guidewire.

13. A device according to claim 12, wherein the at least one hollow receiver member includes a first hollow receiver member and a second hollow receiver member mounted with respect to the elongated probe, each hollow receiver member configured and dimensioned to receive a K-wire or guidewire; and wherein the first hollow receiver member is mounted with respect to a distal portion of the elongated probe and the second hollow receiver member is mounted with respect to a proximal end of the elongated probe.

14. A device according to claim 12, wherein the at least one hollow receiver member extends from a distal portion of the elongated probe to a proximal end of the elongated probe.

15. A system for use in cranial procedures comprising:
a. a device that includes a handle member, and an elongated probe extending from and mounted with respect to the handle member, the elongated probe supporting an ultrasound transducer for use in obtaining ultrasound images for use in determining the location of the elongated probe relative to surrounding anatomical structures or features; and
b. a tubular member releasably supported by the elongated probe.

16. A system according to claim 15, wherein the tubular member is an EVD catheter or a ventricular drain.

17. A system according to claim 16, wherein the EVD catheter or the ventricular drain defines an opening, channel, window or other structural feature that permits unobstructed ultrasound imaging from the ultrasound transducer.

18. A system according to claim 15, wherein the handle member defines a guide channel and wherein the system further comprises a K-wire or guidewire removably positioned in the guide channel.

19. A method for performing a cranial procedure, comprising:
a. providing a device that includes a handle member, and an elongated probe extending from and mounted with respect to the handle member, the elongated probe supporting an ultrasound transducer for use in obtaining ultrasound images for use in determining the location of the elongated probe relative to surrounding anatomical structures or features; and
b. introducing the elongated probe to a desired cranial location while obtaining ultrasound images for use in assessing the position of the elongated probe relative to surrounding anatomical structures or features.

20. A method according to claim 19, wherein the cranial procedure is selected from the group consisting of a cranial aneurysm procedure/surgery, a vascular procedure/surgery, an intra-cranial procedure/surgery, an extra-cranial procedure/surgery, a bypass procedure/surgery and a tumor-related procedure/surgery.

21. A method according to claim 19, further comprising providing a K-wire or guide wire that passes through a guide channel in the handle member and using the K-wire or guide wire to guide one or more further structures to a desired anatomical location.

22. A method according to claim 19, further comprising providing a tubular member and using the elongated probe to deliver the tubular member to a desired anatomical location.

23. A method according to claim 19, further comprising providing at least one of a curette, a probe, a knife, a suction device, a scissor, a cautery unit, forceps and a grasping device for use in conjunction with the elongated probe.
